(19) United States
(12) Patent Application Publication
(10) Pub. No.: US 2008/0090850 A1
(43) Pub. Date: Apr. 17, 2008

(54) Herpetic neuralgia topical treatment

(76) Inventor: Mark E. Anderson, Newport Beach, CA (US)

Correspondence Address: STETINA BRUNDA GARRED & BRUCKER, 75 ENTERPRISE, SUITE 250, ALISO VIEJO, CA, 92656, US

(21) Appl. No.: 11/974,280

(22) Filed: Oct. 12, 2007

Related U.S. Application Data
(60) Provisional application No. 60/851,590, filed on Oct. 13, 2006.

(51) Int. Cl. A61K 31/195; A61K 31/164; A61K 31/52; A61P 25/00; A61K 31/19

(52) U.S. Cl. 514/263.4; 514/561; 514/572; 514/626

(57) ABSTRACT

Compositions and methods for treatment of acute herpetic zoster and post herpetic neuralgia. The compositions comprise the combination of five or six components, namely, a carrier penetrator, an anti-inflammatory agent, a topical analgesic, a nonsteroidal anti-inflammatory penetrator, a direct-application anti-viral agent, and, optionally, an anti-convulsant and calcium-channel blocker. The compositions are formulated to be applied topically to the affected areas.

### CROSS-REFERENCE TO RELATED APPLICATIONS

The present application claims priority to U.S. Provisional Application Ser. No. 60/851,590 entitled Herpetic Neuralgia Topical Treatment filed Oct. 13, 2006, the entire disclosure of which is incorporated herein by reference.

### STATEMENT RE: FEDERALLY SPONSORED RESEARCH/DEVELOPMENT

Not Applicable

### BACKGROUND

Herpetic neuralgia is one of the primary neuropathic pain syndromes in the United States today. Herpetic neuralgia and post-herpetic neuralgia syndrome are very common, affecting approximately twenty percent of the entire population of the United States during a lifetime. There may be as many as one million cases in the United States per year and three million cases worldwide in English-speaking, western, and affluent Asiatic countries.

The varicella zoster virus is generally known to cause two diseases; varicella, commonly referred to as chicken pox, and herpes zoster, commonly referred to as shingles. After an individual has chickenpox, the virus lives in the nerves and is never fully cleared from the body. Instead, the virus retreats to the nerve cells within the sensory ganglion, typically those within the thoracic spine. The virus can lie dormant for several months or years and even up to several decades. Herpes zoster is the reactivation of the varicella zoster virus. This reactivation may lead to a secondary disorder referred to as post-herpetic neuralgia, which is the most common and debilitating sequelae of herpes zoster.

Cell-mediated immunity appears to be the source of reactivation of the varicella zoster virus. Generally, the immune system suppresses the reactivation of the virus. However, a decrease in the immune system response coupled with an immune-compromising event may cause the reactivation. Immune-compromising events can be caused by aging, severe emotional distress, immunosuppression, severe illness, or long-term use of corticosteroids. Therefore, the development of acute herpes zoster is common in elderly individuals who have recently experienced a major life event, such as the death of a loved one, surgery, a major illness, or an associated viral illness. In another common example, an immune-compromised patient, such as a patient with HIV or cancer, or a patient receiving immunosuppressive drugs, such as steroids or chemotherapy, are at increased risk for developing acute herpes zoster.

When the varicella zoster virus reactivates, the virus begins to replicate in the nerve cells of a ganglion and the newly formed viruses are carried down the axons of the peripheral nerve to the area of the skin served by a particular ganglion, destroying tissue and causing demyelinating fibrosis. An intense inflammatory reaction occurs on the affected skin surface in the formation of blisters and within the cutaneous sensory nerves. The virus also travels from the ganglion along the peripheral nerve toward the spinal cord, and in some cases, inflammation in the spinal cord may result. An individual may suffer chronic neuropathic pain due to viral damage to the peripheral nerves and as a result of direct damage to the spinal cord as well. In some cases, severe inflammation, hemorrhage or even necrosis occurs in the affected sensory ganglion, a condition known as ganglionitis.

While it is assumed that all patients with herpes zoster have some degree of nerve damage, some patients, but not all, develop chronic pain or post-herpetic neuralgia. Post-herpetic neuralgia is commonly defined as pain that persists or recurs at least one month after occurrence and subsequent healing of herpes zoster in an individual. This pain includes any pain following rash healing to pain persisting for at least three months after rash healing. Although the specific pain duration that completely distinguishes acute herpes zoster pain from post-herpetic neuralgia remains unknown, the definition of post-herpetic-neuralgia has important implications for interpretation of research findings.

The diagnosis of post-herpetic neuralgia is straightforward. The patient presents with a viral outbreak, consistent with acute herpes zoster in post-herpetic neuralgia. The individual suffers from continued pain in the affected region, which can occur without the development of a rash on rare occasions. However, an acute herpetic outbreak typically manifests itself as an erythematous indurated blister-like outbreak on the skin in a patchy distribution, consistent with dermatomal anatomical distribution. The rash heals in two to four weeks in virtually all immune-competent patients. A definitive diagnosis of the condition is typically made by laboratory testing or evaluation of a serum or spinal fluid of the individual demonstrating evidence of acute herpes zoster infection during an episode of dermatomal pain. Most patients have a gradual resolution of the pain, whereas others continue to experience debilitating pain for months or years. An individual suffering from post-herpetic neuralgia may also develop a deep white discoloration and skin scarring in areas where the herpes zoster rash is severe.

The primary symptom of post-herpetic neuralgia is location. The most common site for post-herpetic- neuralgia is in the thoracic dermatome followed by the VI dermatome, which is the first division of the trigeminal nerve and includes the scalp and forehead. Ophthalmology evaluation is recommended for VI dermatome involvement because possible ocular involvement can result in blindness. Post-herpetic neuralgia and acute outbreak can also occur in the cervical, lumbar and sacral dermatomes.

Another symptom of post-herpetic neuralgia is the spread of pain. The region of suffered pain of an individual having post-herpetic neuralgia may involve several dermatomes unilaterally. However, the region of pain is more often than not restricted to one, or several, regions of the skin where the dermatome is affected by a herpes zoster rash. In some patients suffering from post-herpetic neuralgia, the area of pain may become larger than the original affected area, with pain and skin sensitivity spreading to involve several dermatomes. The spread of pain is believed to be due to central neuroplasticity that is changed in the central nervous system, caused by abnormal output in post-herpetic neuralgia.

Another symptom of post-herpetic neuralgia is the distribution. As with all neuropathic pain in post-herpetic neuralgia, the pain may be described in terms of a variety of sensations which include burning, rawness, sharpness, electric-like, deep aching and freezing cold. Dynamic allodynia is especially common in post-herpetic neuralgia and has been found in approximately eighty percent of patients. Post-herpetic neuralgia patients are also more likely to complain of an acute abnormal itching sensation associated with allodynia, especially when the VI region is affected.

Another symptom of post-herpetic neuralgia is the duration of the pain. In the natural progression of the herpetic zoster virus, the pain is acute and intense within two weeks of appearance of the rash. Pain gradually resolves spontaneously in most patients, but in the elderly, it is more common that the herpetic zoster virus progresses into post-herpetic neuralgia. Once a patient has had post-herpetic neuralgia for one year, it is unlikely that the pain will spontaneously resolve.

Other symptoms of post-herpetic neuralgia include the development of weakness and loss of muscle tone, in addition to tremors and paralysis, if the nerves involved control muscle movement. In addition, an individual may have signs of depression, anxiety and sleep disorder. All of these symptoms should be brought to the attention of a physician to allow for early diagnosis and prompt treatment.

A number of risk factors are identified for development of postherpetic neuralgia. For example, the incidence of herpes zoster in individuals is markedly increased in individuals above the age of fifty. In addition, the risk of post-herpetic neuralgia also increases with age. For example, pain has been found to persist for greater than one year in almost fifty percent of the herpes zoster patients older than age 70. Severe pain during the acute herpes zoster infection, a severe herpes zoster infection or a severe rash associated with the herpes zoster condition are also an indication of the possible onset of post-herpetic neuralgia. The presence of prodromal pain, polyneuropathy, or psychosocial stressors at the onset of herpes zoster are also indicators of an elevated risk that the individual will develop post-herpetic neuralgia.

While no treatment exists for individuals suffering from herpes zoster that will completely prevent post-herpetic neuralgia, a number of known treatments have been found to reduce its likelihood. For example, studies have shown that anti-viral therapy reduces the percentage of patients with herpes zoster who develop post-herpetic neuralgia. These anti-viral agents include acyclovir, famciclovir and valacyclovir. The anti-viral medications must be given early, and within several days of the onset of the rash associated with herpes zoster in order to maximize the benefits. It is noted that such treatments are not one hundred percent effective. Although the probability of post-herpetic neuralgia decreases with the use of anti-viral treatment, early rash resolution is not guaranteed. However, by reducing the severity of the rash and hastening the healing of the acute infection early, the anti-viral treatment is likely to decrease inflammation and minimize the destructive process associated with the varicella zoster virus. Still, viral reaction in the form of ganglionitis can occur prior to the development of the rash and, before anti-viral therapy can be initiated.

Nerve blocks during acute herpes zoster can, in theory, be beneficial. Nerve blocks will reduce the abnormal ectopic discharges from the damaged peripheral nerves and may therefore prevent the development of central nervous system (CNS) changes associated with chronic neuropathic pain. However, no controlled studies are believed to exist that demonstrate the efficacy of nerve blocks. Therefore, it is not clear that nerve blocks can be a preventor of post-herpetic neuralgia although some studies have shown that nerve blocks can be utilized for the performance of severe pain blocking with acute herpes zoster. Other associated adjuvant medications, including anti-depressants and anti-convulsants, may provide preemptive analgesia, but evidence in support of this is limited.

Patients often have extreme sensitivity for areas of the outbreak, and the sensitivity is so great that the areas are difficult to touch or dress and may prevent functional use of the area, as for example, a joint. Thus, oral treatments are generally preferred, and such oral treatments include therapeutic levels of Gabapentin, ranging from 100 milligrams per day up to 6,000 milligrams per day, with the average effective dosage between 2,100 milligrams and 3,600 milligrams per day. The common use of Gabapentin for other applications does not exceed 3,200 milligrams per day. Tricyclic anti-depressants, including amitriptyline and desipramine, have been shown to be beneficial but have substantial side effects, including sedation, dry mouth, postural hypotension, blurred vision and urinary retention, in addition to cardiac conduction abnormalities and liver toxicity. Opioids are often used because of the degree of severity of both acute and chronic pain but the use of opioids often requires around the clock dosing with adjustment for breakthrough pain on an as-needed basis. Opioids are thus difficult to use and have been associated with addiction issues. Muscle relaxants, intravenous lidocaine infusion and oral mexiletine have also been prescribed.

Accordingly, there is a substantial need in the art for the treatment of acute herpetic zoster, as well as post-herpetic neuralgia. There is likewise a need for an effective treatment of such conditions that can be easily and readily administered, and especially administered such that affected areas can be directly treated. There is still further need for an effective treatment of such conditions that is relatively simple formulation, easy to administer and substantially more effective than prior art remedies.

### SUMMARY

The present invention specifically addresses and alleviates the above-identified deficiencies in the art and is directed to compositions and methods for the treatment of both acute herpetic zoster outbreak and post-herpetic neuralgia by the application of a topical medicament. This topical application thereof is proposed despite the sensitiveness of the areas being treated, which sensitiveness would normally urge a different form of treatment.

The pharmaceutical compound is formulated for topical application only and is comprised of five or six components in various combinations and amounts. The first component comprises a carrier penetrator, which may be oil-based in nature and may comprise a fish oil derivative such as Omega-3. The second component comprises an anti-inflammatory agent, which may include a steroid such as cortisone. The third component comprises a topical analgesic, such as xylocaine and other like compounds opioids and oxycodones and several non-opioids made suitable for use as the topical analgesic component. The fourth component comprises a nonsteroidal anti-inflammatory penetrator, which may include aspirin, or, alternatively, other well-known nonsteriodal anti-inflammatory agents such as Ketoprofen, Ibuprofen, Acetaminophen, and the like. An optional fifth component comprises an anti-convulsant and calcium-channel blocker, such as gabapentin. The sixth component comprises a direct-application, anti-viral, such as acyclovir.

The aforementioned components may be combined at ratios relative one another to create compositions of varying degrees of potency. Moreover, such components may be formulated to take a variety of forms well-known in the art, such as aerosol sprays, foams, lotions and any other forms suitable to facilitate the topical application of such compositions. Along these lines, it is contemplated the compositions of the present invention may be delivered through the use of topical transdermal type patches well-known in the art.

### DETAILED DESCRIPTION

The detailed description set forth below is intended as a description of the presently preferred embodiments of the present invention, and is not intended to represent the only form in which the present invention may be formulated or utilized. The description sets forth the functions and sequence of steps of formulating and utilizing the invention. It should be understood, however, that the same or equivalent functions and sequences may be accomplished by different embodiments and that such different, alternative embodiments are expressly intended to be encompassed within the scope of the present invention.

The present invention is directed to compositions and methods for the treatment of acute herpetic zoster outbreak and post-herpetic neuralgia via the application of a topically-applied medicament. Such medicament is comprised of five to six key components in various combinations and amounts, as set forth below.

The first component is a carrier penetrator for the active ingredients. The disrupted vesicularized nerve endings on the cutaneous surface are neural, oil receptors, and therefore provide a direct access into the inflamed neuron end plates. The carrier penetrator may comprise a fish oil derivative, such as omega-3. Omega-3 may be obtained from cold water fish, such as salmon or herring, and is commonly utilized in fish oil tablets. Although omega-3 fatty acid is the preferred carrier penetrator for the active ingredients, other carrier penetrators comprising an oil-based material suitable for absorption by the vesicularized nerve endings on the cutaneous surface may be also be utilized. The carrier penetrator may be optionally accompanied by a tocopherol, such as vitamin E, which additionally provides a component of saponification for topical application to the skin surface by enhancing coating and attachment penetration to the skin surface. The lipophilic membrane transport is preferably achieved by omega-3 fatty acid, which, in turn, is composed of alpha-linolenic acid (ALA), eicosapentaenoic acid (EPA), and docosahexaenoic acid (DHA). In one embodiment, the composition may comprise a ratio of carrier penetrator to saponificant of about 2:1 or, more particularly, about 2 cc of omega-3 and about 1 cc of tocopherol.

The second component is an anti-inflammatory agent, such as cortisone. A three percent cortisone ointment may be utilized in commercial preparation with a petroleumized ointment to provide long lasting application and slow penetration. A total of about 2 cc is preferable for the proposed mixture. If repeated application is not an obstacle to use or is otherwise desired, then the petroleum base can be omitted.

The third component is a topical analgesic, such as xylocaine. Preferably, a five percent xylocaine may be utilized in commercial preparation with a petroleum ointment to provide long-term surface coating and penetration. Xylocaine, also known as lidocaine, is commonly used to provide topical analgesic effects for the high intensity of both acute and chronic pain. However, if the pain is manageable by the patient, this component may be reduced or omitted. Other topical analgesics may be substituted in an appropriate amount for xylocaine, including tetracaine, bupivacaine and benzocaine. Short and long-acting opioids may also be used, including topical application of sustained release morphine, such as MS Contin; oxycodone hydrochloride, such as oxycontin, or fentanyls, such as the Duragesic patch. It is believed that several non-opioids may be used, such as gabapentin, topiramate and lamotrigine. Depending on the particular pain level of the patient and the particular topical analgesic component, the amount of topical analgesia can be varied.

The fourth component is a nonsteroidal anti-inflammatory penetrator, which has a carrier capacity and bonds with and penetrates the neural element. In one embodiment, the nonsteroidal anti-inflammatory penetrator is aspirin, such as 5-grain. However, this component may be substituted by therapeutic levels of ketoprofen, indomethicin, ibuprofen, acetaminophen or diclofenac on a 10% equivalent dose regimen.

The fifth component is an anti-convulsant and calcium-channel-blocker, preferably gabapentin dissolved in the solution following pumice/dilution preparation. An amount of about 10 milligrams of gabapentin dissolved in the solution following pumice/dilution preparation is believed suitable. Gabapentin is a well-known and well-tolerated anti-convulsant that is commonly used as a calcium-channel blocker to decrease neuronal input and decrease the pain transmission from the inflamed nerve affecting a pain blocker agent. This component allows application of low doses and allows direct contact to irritated nerve endings, providing maximum effectiveness with minimum side effects. This component is optional, but preferred. Thus, the predominant application would be with or without gabapentin.

The sixth component is a direct-application anti-viral, such as acyclovir. Acyclovir is anti-viral and required for direct application. The anti-viral is taken up in the raw nerve ending receptor, travels through the axon of the nerve to the ganglion and results in direct treatment of the infected viral ganglion. The anti-viral is selected to provide the combination of highest dose concentration while providing the minimal side effect when directly applied onto the inflamed skin area and associated nerve endings. One particular application may include 400 mg/5 cc, or 80 mg/cc. Other anti-virals may be employed at other dosages and depending on the efficacy and resulting side effects.

For treatment, a 10 cc application can be prepared in accordance with the above discussion of the various components. The above components can be combined in appropriate ratios to produce different volumes of material.

The application treatment pattern comprises one application every six to twelve hours with a maximum of four applications per day in any form. Presently, it is contemplated that therapeutically effective amounts of the compounds utilized in the practice of the present invention can comprise anywhere between approximately 1 milligram to 1 gram of such compound per square centimeter of the affected area of the subject. The duration of treatment is estimated to be two to four weeks in the acute phase and up to one year in the chronic or post-herpetic neuralgia phase.

Although the above combination results in an oil-base product, there are many variations, which can include aerosol spray and foam of the mixture of the above components, suitably modified to delete the petroleum and ointment base and the other components adapted for aerosol spray or foam applications. The components could also be modified for application in a topical liquid other than oil. A long-term coating application with overlying protective foam which solidifies is also believed suitable.

In one embodiment, the composition may be placed in an apparatus containing an amount of the composition; a vessel containing the composition and a dispensing means in fluid communication with the vessel so that the composition may be administered therefrom. The apparatus may be adapted to spray the composition and may be a container fitted with a mechanical pump or a container pressurized with a propellant, which may be a conventional aerosol propellent such as nitrogen or a hydrocarbon. The apparatus may be further adapted to discharge a metered dose of the composition on actuation.

The composition may also be prepared in the form of a gel, cream, lotion, paste or ointment. The composition may be dispensed from a squeeze-type tube or a plunger type applicator. The composition may also be impregnated on bandages, treatment patches, such as topical transdermal type patches, or cloth wipe products.

Multiple variations and preparations of the above combinations can be used. These include all physiological dose regimen combinations. The pharmaceutical compositions described herein can be manufactured in a manner that is known to those of skill in the art by conventional mixing, dissolving, and/or emulsifying processes. Also, it will be understood that the pharmaceutical compositions suitable for use in the present invention include compositions wherein the active ingredients are contained in a therapeutically effective amount. The amount of composition to be applied is, of course, be dependent on the subject being treated, the severity of the affliction, the manner of topical administration, and the judgment of the prescribing physician. Determination of an effective amount and frequency of administration is well within the capability of those skilled in the art, especially in light of the detailed disclosure provided herein.

The above proposal is for a prescription-only therapeutic component compound given under physician direction by those skilled in the art of acute neurological, infectious disease or pain management. Therapeutic dose ranges of all components are contemplated for topical cutaneous application. A known allergy to any of the components is a contraindication for application.

The above description is given by way of example, and not limitation. Given the above disclosure, one skilled in the art could devise variations that are within the scope and spirit of the invention disclosed herein, including various ways of deriving formulations of the compositions of the present invention for use in topical applications. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A composition for topical cutaneous application, the composition comprising:
a carrier and neuron penetrator for nerve endings on a cutaneous surface in an amount sufficient to dissolve or carry the remaining components of the composition;
an anti-inflammatory in a therapeutic effective amount;
a topical analgesic in a therapeutic effective amount;
a non-steroidal anti-inflammatory penetrator in a therapeutic effective amount;
a direct application anti-viral in a therapeutic effective amount.

2. The composition of claim 1, further including an anti convulsant and calcium-channel blocker in a therapeutic effective amount.

3. The composition of claim 1, wherein the carrier and neuron penetrator further comprises a tocopherol.

4. The composition of claim 3, wherein the tocopherol further includes a saponificant.

5. The composition of claim 4, wherein the ratio of carrier and neutron penetrator to saponificant is 2:1.

6. The composition of claim 1, wherein the carrier and neuron penetrator comprises a fish oil derivative.

7. The composition of claim 6, wherein the carrier and neuron penetrator comprises omega-3.

8. The composition of claim 1, wherein the anti-inflammatory comprises cortisone.

9. The composition of claim 8, wherein the anti-inflammatory is a 3% by weight cortisone preparation in petroleum.

10. The composition of claim 1, wherein the topical analgesic is selected from the group consisting of xylocaine, tetracaine, marcaine, benzocaine, a topical opioid, and a topical non-opioid.

11. The composition of claim 10, wherein the topical analgesic is a 5% by weight xylocaine preparation in petroleum.

12. The composition of claim 10, wherein the topical opioid is selected from the group consisting of MS Contin, oxycodone hydrochloride or fentanyl.

13. The composition of claim 10, wherein the topical non-opioid is selected from the group consisting of gabapentin, topiramate, and lamotrigine.

14. The composition of claim 1, wherein the non-steroidal anti-inflammatory penetrator is selected from the group consisting of aspirin, ketoprofen, indomethicin, ibuprofen, acetaminophen and diclofenac.

15. The composition of claim 2, wherein the anti-convulsant and calcium-channel-blocker comprises gabapentin.

16. The composition of claim 1, wherein the direct application anti-viral comprises acyclovir.

17. The composition of claim 1, wherein the composition is placed in a spray device that on actuation discharges a metered dose of the composition.

18. The composition of claim 1, wherein the composition is in the form of one of a foam, an aerosol, a cream, an ointment, a gel, a paste and a lotion.

19. The composition of claim 1, wherein the composition is impregnated on one of a bandage, a transdermal patch and a cloth.

20. A method of treating post-herpetic neuralgia comprising topically administering to an individual to whom such treatment is needed a combination comprising (a) a carrier and neuron penetrator for nerve endings on a cutaneous surface in an amount sufficient to dissolve or carry the remaining components of the composition; (b) an anti inflammatory in a therapeutic effective amount; (c) a topical analgesic in a therapeutic effective amount; (d) a non-steroidal anti-inflammatory penetrator in a therapeutic effective amount; (e) a direct application anti-viral in a therapeutic effective amount.

21. The method of treating post-herpetic neuralgia of claim 20, wherein the combination further comprises an anti convulsant and calcium-channel blocker in a therapeutic effective amount.

22. A method for treatment of post-herpetic neuralgia, the method comprising the steps of:
preparing a composition containing (a) a carrier and neuron penetrator for nerve endings on a cutaneous surface in an amount sufficient to dissolve or carry the remaining components of the composition; (b) an anti inflammatory in a therapeutic effective amount; (c) a topical analgesic in a therapeutic effective amount; (d) a non-steroidal anti-inflammatory penetrator in a therapeutic effective amount; (e) a direct application anti-viral in a therapeutic effective amount; and
topically applying the composition to the skin of an individual.

23. The method of treating post-herpetic neuralgia of claim 22, where the combination further comprises an anti-convulsant and calcium-channel blocker in a therapeutic effective amount.
